(19) United States
(12) Patent Application Publication
(10) Pub. No.: US 2021/0401288 A1
(43) Pub. Date: Dec. 30, 2021

(54) Non-Invasive Convergent Heating Treatment

(71) Applicant: Michael D. Weil, Half Moon Bay, CA (US)

(72) Inventor: Michael D. Weil, Half Moon Bay, CA (US)

(73) Assignee: Michael D. Weil, Half Moon Bay, CA (US)

(21) Appl. No.: 17/359,869

(22) Filed: Jun. 28, 2021

Related U.S. Application Data
(60) Provisional application No. 63/046,428, filed on Jun. 30, 2020.

(51) Int. Cl. A61B 5/00; A61B 5/0507; A61N 5/02; A61B 5/05; A61N 1/40; A61N 5/06; A61N 1/28; A61B 8/08; A61N 7/02; A61G 15/02; A61N 5/01; A61G 13/02; A61B 8/00; A61N 5/04; A61N 1/20

(52) U.S. Cl. CPC A61B 5/0035; A61N 2005/067; A61N 5/025; A61B 5/05; A61N 1/403; A61B 5/0064; A61N 5/0625; A61N 1/28; A61B 8/5261; A61N 7/02; A61B 5/704; A61G 15/02; A61N 5/01; A61G 13/02; A61B 5/0036; A61B 8/40; A61B 5/6803; A61N 5/04; A61N 5/0622; A61N 5/0618; A61N 5/022; A61N 1/205; A61N 5/0624; A61B 5/0507

(57) ABSTRACT

The invention relates to medical therapy of detrimental lesions. A system treats with non-invasive arrays of non-ionizing energy-radiation sources. The external sources focus energy dose distribution to a selected volume in a body. Energy output is directed towards a pathologic location and quenched around other sites or healthy tissue. Beam aiming is done without source movement. Targeted, volumetrically discrete energy deposition can beneficially manipulate lesions within the body. The purpose of the focused and enhanced energy deposition is to repair or disable pathologic physiology or structures, nerve pathways, extracellular fluid, and misfolded proteins. Locally augmented energy is used to enhance immunity, release pharmaceutical compounds from energy-sensitive vesicles and reconfigure or eliminate misfolded proteins and their aggregates. Targeted tissue may have enhanced sensitivity to received energy via a non-ionizing-radiation, treatment-localizing agent. This system optimizes delivery of non-ionizing, non-ablating electromagnetic or mechanical energy, degrading or repairing an abnormality upon interaction with it.

### RELATED U.S. APPLICATION DATA

This application claims priority of provisional application 63/046,428 with a filing date of Jun. 30, 2020.

### REFERENCES CITED

### U.S. Patent Documents

U.S. Pat. No. 7,481,758 January 2009 Weil and Morris

### OTHER PUBLICATIONS

Crespo et al., “Gene regulatory network analysis supports inflammation as a key neurodegeneration process in prion disease,” BMC Syst Biol 6-132, 2012, BioMed Central.
Dou et al., “To heat or not to heat: Challenges with clinical translation of thermosensitive liposomes,” J. of Controlled Release 249:63, 2017, Elsevier.
Goedert. “Alzheimer's and Parkinson's diseases. The prion concept in relation to assembled Aβ, tau, and α-synuclein,” Science 349(6248):1255555, 2015, AAAS.
Whittington et al., “Hypothermia and Alzheimer disease neuropathogenic pathways,” Cuff Alzheimer Res 7(8):717, 2010, Bentham Science.

### FIELD OF THE INVENTION

The claimed invention relates generally to medical therapy and imaging of lesions that are detrimental to a body using externally applied energy with a localization-enhancing agent. The system requires treatment and imaging with a source to transfer energy within a body. A localizing agent may enhance therapeutic and repair mechanisms.

### BACKGROUND OF THE INVENTION

Inflammation and Fever. Fever is a common symptom of immune response to infection and inadequate cooling. It is a systemic response to what may be a systemic or localized instigating factor. Since temperature maintenance of the central nervous system (CNS) and body within a narrow range is vital for survival, fever likely has natural selection benefits. However, the scope of the induced functions and evolutionary advantages of warming stresses are uncertain. On a molecular level, warming increases kinetic energy disrupting hydrogen and nonpolar covalent bonds, as well as denaturing proteins. High temperature reduces replication of influenza virus, raising endosomal pH and inhibiting IL-6, a mediator of viral reproduction. In the cell signaling realm, modest temperature upticks induce the production of molecular chaperones, such as heat-shock proteins (HSPs) and small heat-shock proteins (sHSPs), to maintain protein conformation and enhance antigen presentation to the immune system. In addition to heat, these chaperones or auxiliary proteins can also be induced by other stresses such as pH or osmotic changes, oxidative or hypoxic fluctuations, interactions with free radicals, aging and protein aggregation disorders, which may be associated with protein denaturation. The chaperones bind to partially unfolded proteins (without requiring ATP) and stop irreversible clumping or aggregation. HSPs are critical in maintaining protein homeostasis (also called proteostasis) and can have anti-apoptotic and anti-inflammatory functions. Thus, they are vital in protein maintenance, repair and degradation. While HSPs stabilize other proteins undergoing stress-induced denaturing influences and conformation changes, they are also critical to immunity. They bind antigens prior to interaction with dendritic cells (antigen presenting cells, APCs) and play an important role in subsequent antigen presentation to T-cells in the lymph nodes during immune activity, as well as during protein repair and removal. Moreover, HSP induction is associated with secondary effects, which are vital for survival. This survival response can be hijacked by infectious agents and cancer cells to make them more lethal. Retrieved from the Internet <URL: https://www.cell.com/cell/fulltext/S0092-8674(07)00957-9?_retumURL=https %3A%2F%2Flinkinghub.elsevier.com%2Fretrieve%2Fpii%2FS0092867407 009579%3Fshowall%3Dtrue> The primary regulator of the effect is heat shock factor (HSF1) and its presence or upregulation is common in malignant transformation. In addition, the Hsp70 promoter when heated can initiate global gene expression in a temporal manner. Beyond HSP induction (with subsequent ameliorative effects on protein structure, function and aggregation), physiologic responses to environmental stressors, e.g., antimicrobial initiated inflammation, can also include generating deep and superficial vasodilation with increased blood flow and localized thermal effects, i.e., heating or cooling. This also results in enhanced oxygenation and immune cell trafficking. However, it seems the primary feedback response to fever-inducing factors is the diversion of circulation towards more cutaneous blood flow to primarily cool the core and CNS temperature. Some of these thermally dependent reaction rates have been modeled by the Arrhenius and Eyring equations.

Inflammatory responses can take place when immune cells or the nervous system typically react to foreign, non-self antigens present on microorganisms, viruses, bacteria and prions, though prions do not induce fever. But, inflammation can develop in the absence of infection and may be restricted to particular locations. For example, gout is a sterile inflammatory disease caused by elevated levels of uric acid, which acts as a local danger signal to stimulate both innate and adaptive immune responses in the face of cellular damage. Such signaling may simultaneously avert activation of autoimmunity (reaction to self-antigens) and associated detrimental effects. In people with gout, it is well-accepted that there is a low rate of multiple sclerosis (MS), which is thought to be an autoimmune disease. Contrariwise, MS patients are intolerant to fever or other heat, which might indiscriminately upregulate (per above) all forms of immunity and thereby worsen their condition.

Systemic body heating may also be induced by cellular inflammatory factors, e.g., cytokines, including but not limited to, interleukin-1 (IL-1), tumor necrosis factor-alpha (TNF-alpha), interleukin-6 (IL-6) or Toll-like receptor (TLR) ligands, granulocyte-macrophage colony-stimulating factor (GM-CSF), cyclooxygenase-2, prostaglandin E2 (PGE2) and activation of hypothalamic PGE2 receptors, pyrogens, e.g., lipopolysaccharides (LPS) and other microbial products, as well as central nervous system (CNS) activity. In preclinical experiments, rats injected with fever-inducing cytokines were unable to generate fevers if the vagus nerve was cut. This may be due to loss of neural signaling to immune cells.

Historically, the average normal human-body temperature was 37 degrees Celsius (98.6 degrees Fahrenheit). However, recent studies concluded the average oral temperature of people has dropped and is now 36.39 degrees Celsius (97.5 degrees Fahrenheit). Retrieved from the Internet <URL: https://elifesciences.org/articles/49555#:˜:text=ln%20the %20US%2C%20the%20normal,temperature%20has%20decreased%20over%20time.> The average core temperature of people over age 60 is approximately 0.4° C. less than healthy younger adults. Retrieved from the Internet <URL: https://www.tandfonline.com/doi/full/10.1080/23328940.2015.1096438> Normally body heat is produced by constitutive metabolism, where the resting metabolic rate is the biggest contributor to energy use (approximately ⅔ of the total). Over time, people's mass has increased, which would be expected to raise body temperature. Therefore, the decrease in body temperature is thought attributable to decreased metabolic rate due to fewer chronic infections, e.g., better dental hygiene, treatment for tuberculosis, more aspirin use, etc., and less chronic inflammation.

Nowadays, fevers are tightly controlled with drugs, such as acetylsalicylic acid (ASA) and acetaminophen. Moreover, fever-inducing infectious disease, e.g., bacteria and biofilms, mycoplasma and viruses, are treated with antibiotics and antivirals. Therefore, in the present era, people likely experience fewer and shorter exposures to fever and physiologic heat stress in their lifetime than their ancestors. It is unknown whether this has an impact on health.

Typically most people now live in environments with automatically controlled ambient air temperature and thus are exposed to less environmental thermal stresses. Unanticipated and unprepared cold exposure is less common than in the past. Following cold exposure, brown adipose tissue generates futile energy consumption cycles, which waste calories to release heat. This thermogenesis in brown adipose tissue depends on the breakdown of branched chain amino acids (BCAA), where brown fat is the primary site of BCAA catabolism.

In addition to fever, inflammatory events and brown fat thermogenesis, there are external, therapeutic methods that can transfer thermal or mechanical energy and be used to heat a body, organ or tissue, which include but are not limited to electromagnetic and mechanical radiations, comprising microwave (MW) radiation, radiofrequency (RF) radiation, long radio waves, ionizing x-rays, α-, β- and □-rays, laser emissions, visible, ultraviolet and solar light, infrared and far infrared light, thermal chambers, or incubators, diathermy or high-frequency electric currents, e.g., short wave or alternating currents, pulsed, mechanical or acoustic waves, ultrasound, high-intensity focused ultrasound (HIFU), electron beams, heat from nano-devices, warm water and other liquids, feeding and nutrients, such as fit, proteins, carbohydrates and alcohol. The foregoing can be delivered via ingestion, topical, intravascular, intrathecal or intraperitoneal routes or baths, hot water blankets, steam, saunas, electrical resistive heating, electric blankets, radiation, induction, magnetic hyperthermia, conduction, convection, insulation, layering, friction, exothermic chemical reactions, combustion and fire. The foregoing technologies can be employed invasively or non-invasively, alone, superficially or deep, centrally or locally, in combination, in arrays, phased arrays or matrices, symmetrically or asymmetrically, with linear or annular setup, stationary, moving, with and without image guidance, with and without artificial intelligence guidance.

Monitoring of fever and external heat application to a body, organ or tissue can be performed with non-invasive or invasive thermometry, in 2-D or 3-D. Measurements can be obtained locally, or systemically, orally, from the axilla, intraperitoneally, rectally, or via intraesophageal data collection. Such devices include, thermometers, thermocouples, thermistors, resistance temperature detectors, pyrometers, fiber optic sensors and infrared. Non-invasive thermometry can be performed with magnetic resonance imaging (MRI), e.g., proton resonance frequency shift, frequency adjustable magnetic fields and optical fiber sensors, computed tomography (CT) and echotomography.

The Nervous System. The central nervous system (CNS) includes the brain, cranial nerves, eyes and spinal column with its associated sympathetic and parasympathetic ganglia and neurons. It is subject to deterioration from normal aging, environmental stresses and a range of pathologies, many of which have unknown etiologies and mechanisms. Possible destructive mechanisms and pathologic lesions, which may be beneficially treated by the present invention, include, but are not limited to, infectious agents, e.g., viruses, bacteria, mycoplasma, fungi, amoebae, prions and protein misfolding or unfolding abnormalities in the proteostasis network (PN), which consists of a thousand-odd components enabling cells to maintain proteins in the proper configuration and are involved in the synthesis, folding, and clearance of proteins (constituting the folded, native proteome); physical and environmental agents and stresses such as lead, temperature, pH and oxidative imbalances; cancer, malignant growths, excess cell proliferation, cancerous and benign tumors and mass effects, demyelination, autoimmunity, loss of immunity, loss of normal mitochondrial function and other organelle function; loss of remodeling and normal cellular pathways; genetic abnormalities, loss of neurotransmitters, loss of neurotransmitter-degrading enzymes, loss of synaptic and cellular receptors, vascular abnormalities and abnormal cerebral blood flow, e.g., including but not limited to aneurysms, atherosclerosis, arteriosclerosis, hemorrhage, blood clots, autoimmune vasculopathy, microvascular angiopathy, retinopathy, excess hemoglobin A1c, diabetic angiopathy; abnormalities of the cerebrospinal fluid (CSF), including its production and movement; hormonal imbalances, trauma and traumatic encephalopathy; mental illnesses such as neuroses, addiction, depression, mania, schizophrenia and psychoses. Since so little is understood in detail about the normal and abnormal function of the central nervous systems, or its structural and mechanistic biology, normal and pathologic classifications are remanded to descriptions of damaged anatomy or function. Moreover, potential treatment interventions have been limited based on access to the components of the CNS. Thus, therapy may involve intracranial structures, extracranial fibers or a combination thereof. However, the exact mechanism of most of the normal and abnormal functionality of the nervous system is restricted to high-level representation. For example, diseases are described by their symptoms, such as motor function disorders, and cognitive impairment. And, although the vulnerable processes of CNS disease remain elusive, therapeutic drug and device applications might nevertheless be derived from existing knowledge and clinical observations.

Pathologic review of patients with Alzheimer's Disease (AD) demonstrates aggregates of amyloid-beta (AP) and neurofibrillary tangles in the brain. The structure of the pathologic aggregates and how they cause disease are unknown. Two proteases, β-secretase (BACE-1, β-site APP-cleaving enzyme) and □-secretase, cleave amyloid precursor protein (APP) to release Aβ. It is thought Aβ clumps initiate spreading of tau neurofibrillary tangles and neurologic deterioration with memory impairment. Monoclonal antibodies directed against Aβ have had only modest impact on amyloid and minimal clinical effects on symptomatic AD. Likewise, reduction in Aβ levels via □-secretase inhibitors, immunotherapy and a recent trial of BACE-1 inhibition have not mitigated AD progression. In addition to extracellular Aβ peptides, aggregated proteins seen with AD include intracellular assemblies of tau proteins, which are predominantly expressed in neurons, especially those of the CNS. Normal tau binds and stabilizes microtubules and has a role in cytoskeletal remodeling important for neuronal plasticity and memory functions of the hippocampus. Tau can be phosphorylated (p-Tau) and form inclusions, which are commonly found in high levels in AD and other neurodegenerative pathology. Misfolded tau proteins form hyperphosphorylated aggregates in neuronal cytoplasm. Such hyperphosphorylated tau is associated with destabilized microtubules. Short fibrils can spread to other cells, i.e., extending tau aggregates through the brain. Hypothermia and reduction in core temperature, as happens for example with systemic anesthesia, increases levels of p-Tau (Whittington et al., “Hypothermia and Alzheimer disease neuropathogenic pathways,” Curr Alzheimer Res 7(8):717, 2010, Bentham Science). There is an 80% increase in p-Tau for each degree Celsius below normothermia. Cold-induced p-Tau also occurs in hibernating and non-hibernating animals. In experimental models, p-Tau elevation could be undone by restoration of core temperature. It has been postulated that cold inhibits proteasomes and leaves p-Tau to amass. Retrieved from the Internet <URL: https://www.tandfonline.com/doi/full/10.1080/23328940.2015.1096438> Mechanism-based approaches have been used to develop compounds to restrict Aβ and tau assemblies or stimulate their destruction by proteasomes and enzymes with limited success. Generally, proteins tolerate a range of temperatures, pH and pressure. Stability differences derive from the energy needed to unfold a given protein consistent with the “principle of minimal frustration.” Retrieved from the Internet <URL: https://www.pnas.org/content/114/9/E1627.short> However, the process of the temperature variation of p-Tau accumulation, and whether it results from functionality similar to thermal-sensitive proteins, is unknown. In comparison, there are temperature-sensitive (ts) proteins with mutations, which cause them to lose function at non-permissive, higher temperatures due to unstable folding. Their heat sensitivity is seen above the restrictive temperature. On the other hand, cold-sensitive (cs) mutant proteins lose function below a cutoff temperature, and at higher temperatures behave like wild-types. At their permissive temperatures, they likewise fold and function properly.

Other neurodegenerative diseases, such as Parkinson's disease (PD), a movement disorder with increased incidence with aging, may share similar pathologic molecular mechanisms seen in AD. Retrieved from the Internet <URL: https://science.sciencemag.org/content/349/6248/1255555> Like AD, PD is thought to be caused by a small number of aggregated proteins. Mutations in the tau gene (microtubule-associated protein tau, MAPT) produce dominant inheritance of frontotemporal dementia (without Aβ plaques) and PD. Other examples of so-called tauopathies include: frontotemporal dementia with PD linked to chromosome 17 (FTDP-17), Pick's disease, corticobasal degeneration and progressive supranuclear palsy. In addition to tau, PD predominantly demonstrates intracellular inclusions of the protein α-synuclein (from the SNCA gene) in Lewy neurites and Lewy bodies.

Though aggregated proteins may be important underlying mechanisms of CNS diseases, the prion-like classification has not usually been invoked beyond person-to-person transmissible diseases, e.g., Kuru, Creutzfeldt-Jakob disease (CJD). Though transmissible proteins may enable normal biologic processes, normal prions, PrP-sen or PrP(C), exist in brain neurons and seem to play a role in maintenance of myelin, circadian rhythms, sleep and neuronal excitation. Moreover, PrP(C) is found on the cell surface and commonly in immune-privileged organs, e.g., brain, eye, placenta, pregnant uterus and testes, where it modulates and is protective against inflammatory stress. Phenotypic changes from PrP(C) loss are minor under normal conditions, but obvious under stress or inflammation in the immune-privileged sites.

Transmissible proteins in the misfolded version form β-sheet-rich aggregates. When PrP(C) is mutated and abnormally folded in β-sheet-rich conformation, known as PrP-res or PrP(Sc), it can interact and convert PrP(C), thus multiplying and condensing into toxic strands that destroy neurons and generate spongiform defects. After oral intake, infectious prions can be found in the Peyer's patches of the small intestine, as well as the enteric nervous system. Airborne prions can directly invade the nervous system in experimental mice and cause scrapie. Prions are resistant to ionizing radiation and conventional sterilization methods. Neuroinflammation plays a key role in initiating prion disease as well as PD and AD; though they do not usually generate a fever, AD may be associated with a slightly higher core temperature.

Prion functionality may be relatively common and at work in multiple neurodegenerative diseases associated with abnormal protein assemblies. Many such illnesses now seem due to particular protein misfolding, aggregation, and accumulation. Misfolded protein aggregates are involved in >20 human disorders, labeled protein misfolding disorders (PMDs), and include AD, PD, and type 2 diabetes. Although the proteins and symptoms associated with these illnesses differ, they have similar reliance on protein misfolding processes. Of note, recent discovery that these misfolded aggregates self-propagate by seeding, and thereby spread pathologic functions between cells, mirrors infectious prions and prion disease mechanics. It has been proposed that pathological peptide-germs begin via exposure to amide units. Once initiated, molecules alter shape then join and expand aggregates. Fragmentation creates additional aggregates. Unbranched filaments grow to ˜10 nm diameter and a length of several micrometers. The purpose of the focused and enhanced non-ionizing energy deposition of the present invention, wherein energy output is effectively directed towards a desired location, specific or selected volume, is to reverse or rectify prion-like mechanisms, wherein misfolded, or unfolded proteins, which may be unaggregated or aggregated, are subjected to a permissible temperature, or energy dose, and thereby enable restoration to their native, normal conformation. Thus, as taught in the present invention, emergence of a pathologic state resulting from peptide clustering parallels dynamic systems where phase transition takes place when control parameters cross critical thresholds. This invention further recites that an original condition, state, conformation, functionality or phase in a body may be obtained by introducing adequate localized energy to a deleterious altered process, network or system, and thereby overcoming an undesired transition. For example, impending transitions due to broken symmetry or dynamic instability can be reversed by externally applied targeted energy to modify and reset control parameters such as attenuating density or virality. Thereafter, a phase transformation can be slowed and a phase restored by permissive and enabling forces. In the present invention, the phase refers to a state in the body.

### SUMMARY OF THE INVENTION

This invention entails optimized therapeutic modalities for the CNS and body that can locally deliver different energy and forces. This may include, but is not limited to, the delivery of electromagnetic energy, thermal energy or heat, or the delivery of mechanical energy or pressure, to affect proteins, nucleic acids and genetic matter, cells and subcellular structures, intra- and extracellular pH, fatty layers, and the extracellular matrix (ECM). Moreover, targeted delivery of energy or forces within the CNS and body can be used to deploy and enhance other treatment modalities, such as elemental and molecular compounds, nanoparticles, antibodies, porphyrins, lipid rafts and proteins. On a molecular level, local energy delivery, such as warming, increases kinetic energy disrupting hydrogen and nonpolar covalent bonds. Pressure changes can vaporize or tear structural molecules. The therapeutic mechanisms may in addition be the result of the expression of HSPs, damage signals, neoantigens, immunogenicity and immunity, optimization of the blood-brain barrier and its permeability, rectification of prions and prion-like peptide molecules, quorum sensing, or optimizing drug delivery or harmonic frequencies and magnetic fields.

Blood-Brain Barrier and Microglia. It is an object of this invention to provide a method to better manipulate the blood brain barrier (BBB) as well as reduce entangled proteins and plaque burden. In a transgenic mouse model, increased deposits of Aβ and memory impairment, can be normalized by treatment with scanning ultrasound, e.g., single spot or sequentially moving with microbubbles (5 times over 6 weeks, 0.7-MPa peak rarefactional pressure, 10-Hz pulse repetition frequency, 10% duty cycle and 6-second sonication time per spot). Retrieved from the Internet <URL: https://stm.sciencemag.org/content/7/278/278ra33> It was thought that the process opened the BBB and microglial cells were activated to take up the Aβ into their lysosomes. This approach modestly reduced Aβ and plaque burden (unless repeated wherein it was more effective). It also improved recognition memory and hippocampal-dependent spatial learning. It was considered to be about as effective as passive Aβ immunization. It is noteworthy though that these animal models lack the full range of AD pathophysiology. Moreover, a human brain and skull are much larger than a mouse's.

CSF and Blood Flow. CSF flows are coupled to blood-flow and blood-oxygenation changes during slow-wave (non-REM, <4 Hz) sleep. These effects are thought to be driven by the slow-wave electrical activity where associated reduced blood volume in the brain results in increased CSF volume. Retrieved from the Internet <URL: https://science.sciencemag.org/content/366/6465/628> The pulsatile flow of CSF into and out from the brain, ventricles and meninges removes toxic waste by mixing with the interstitial fluid of the CNS. But, slow-sleep is disturbed with aging and in Alzheimer's disease. Thus, potentially toxic proteins, e.g., amyloid-beta (Aβ), tau, might not be effectively cleared during sleep. This invention includes heating the CSF, thereby producing waves of flow, and enhancing its flow around and interaction with the brain can be employed to reduce toxic or waste products. Such heating might result from fever or by external induction as noted above, e.g., microwaves or mechanical pressure waves, in the present invention. The external energy of the arrayed microwave delivery system of the present invention may also enhance the permeability of the blood-brain barrier to allow entry of beneficial molecules and removal of toxic by-products. Localized energy delivery, such as heating, may enhance the solubility of proteins such as tau proteins, which are abundant in the neurons of the CNS. In this way, even prion-like aggregates of mutated or damaged proteins may be reduced or reversed or eliminated.

Microbiome, Autism and Tau. It is another object of this invention to provide a method to manipulate the microbiome and altered intestinal barriers. The brain and CNS are also profoundly influenced by the microbiome, i.e., the microorganisms in the body and their genetic material and byproducts. In one example, the microbiome of the gut has been shown to result in neurological development disorders. Retrieved from the Internet <URL: https://www.nature.com/articles/s41598-019-42183-0> People with Autism Spectrum Disorders (ASD), Parkinson's disease (PD) and Alzheimer's disease have chronic gastrointestinal conditions, which suggest a gut-brain axis of interaction. In fact, transfer of commensal gut bacteria from healthy donors has been shown to transform dysbiotic gut microbiomes and improve autistic behavior in children with ASD. Such microbial transfer therapy (MTT), has been performed after oral antibiotics, bowel cleanse, and oral antacids prior to microbiota transplant. The population diversity of the microbiome in the gut, and other anatomy, can be driven towards a healthful profile by energy deposition, including heating, in the locations harboring them. Thereby, the neurological and muscular disorders and the symptoms of the neurocognitive and neurofunction pathologies might be treated as taught in the present invention. Retrieved from the Internet <URL: https://www.ncbi.nlm.nih.gov/pubmed/18509436>

DNA from gram-positive and gram-negative bacteria increases tau misfolding. This effect is more pronounced with DNA from microbes found in the CNS or mouth of AD patients. The compromise of blood-brain and intestinal barriers, might act as significant sources of microbial DNA in the CNS. Any resulting augmentation of tau misfolding, and thereby AD pathogenesis, can be targeted by way of the recitations of the present invention.

Vagus Nerve (X) Stimulation. This invention provides a more effective and less toxic method for targeting interactions between the nervous and immune systems. The use of pulse generators surgically implanted next to the left or right vagus nerve, the tenth (X) cranial nerve, has been reported to improve epilepsy, depression, rheumatoid arthritis and heart disease. The vagus nerve, by way of its afferent fibers, relays critical physiologic performance from the lungs, heart, gut and liver to the CNS. It is thought that the gut's microbiome may influence the brain and immune system via this route. In tandem, the brain seems to influence the spleen and other components of the immune system via the vagus nerve's extracranial fibers and efferent signaling, e.g., cholinergic mechanisms. This signaling, acting through beta-2 adrenergic receptor (□₂-AR) agonists in splenic T-cells, and alpha-7 nicotinic acetylcholine receptors (α7-nAChR) in splenic and intestinal macrophages and dendritic cells, is reported to be important for anti-inflammatory pathways. Of note, both agonists and antagonists of α7-nAChR have been reported to show efficacy in models of Alzheimer's Disease (AD) and other cognitive disorders, as well as inflammatory diseases. Retrieved from the Internet <URL: https://www.ncbi.nlm.nih.gov/pmc/articles/PMC3292018/> The release of such neurotransmitters as part of the inflammatory reflex is thought to thereby link metabolism to immunity. The present invention includes non-invasive targeting of the vagus and other cranial nerves, such as the olfactory and ophthalmic nerves and eyes, with MW and ultrasound energy to modify the inflammatory reflexes linking metabolism to immune function.

Other Effects of Oscillating Energy. In another aspect, this invention includes a safer, directed method to deliver oscillating energy to treat functional and mechanical characteristics of pathology. Correlation between reduced gamma oscillations (20-50 Hz) and cellular dysfunction has been reported in neurologic disease though has not been well characterized. Investigations in an ontogenetically engineered mouse model, used flickering visible light, sound alone and combinations thereof to generate and drive gamma oscillations (40 Hz) to ameliorate AD findings, and reduce Aβ and phosphorylated tau. Retrieved from the Internet <URL: https://www.nature.com/articles/nature20587>, Retrieved from the Internet <URL: https://www.ceIl.com/cell/fulltext/S0092-8674(19)30163-1>.

In oncology, low intensity (I_{SPTA}<5 W/cm2), pulsed ultrasound can selectively ablate cancer cells, leaving normal cells unharmed. Ultrasound at a frequency of 0.5-0.67 MHz and a pulse duration of >20 ms forms acoustic standing waves with cell-centered cavitation and cell death specifically for cancer cells. Retrieved from Internet <URL: https://aip.scitation.org/doi/10.1063/1.5128627>. Presumably, the ultrasound is selective for the mechanical properties of the malignant cells. This notion that cancer cells are vulnerable, e.g., the cellular skeleton breaks down, to specific frequency of ultrasound has been termed “oncotripsy.” Retrieved from the Internet <URL: https://www.eurekalert.org/pub_releases/2020-01/aiop-usd010320.php>.

A device, NovoTTF-100A, applying alternating electric fields (100-500 kHz, 1-3 V/cm) via overlying electrodes to treat cancer is marketed in the U.S. (NovoCure, Haifa, Israel). Likewise, pulsed-electric fields, electroporation techniques, have been used to increase cell membrane permeability and thereby enhance uptake of chemotherapy and cell death. Retrieved from the Internet <URL: https://www.ncbi.nlm.nih.gov/pubmed/21812011>.

Amplitude-modulated radiofrequency electromagnetic fields (AM RF EMF) affect voltage-gated calcium channels in treating hepatocellular cancer (HCC) at “tumor-specific” frequencies and can have antitumor effects without inducing hyperthermia or normal tissue damage (TheraBionic GmbH, Ettlingen, Germany).

### BRIEF DESCRIPTION OF THE FIGURES

The construction and usage of embodiments will become readily apparent from consideration of the following specification as illustrated in the accompanying drawings, in which like reference numerals designate like parts, and wherein:

FIG. 1 is a diagram illustrating a system according to some embodiments, which stands alone and treats the head and CNS;

FIG. 2 is a simplified perspective view of arrayed microwave antennas and delivery components according to some embodiments, which stands alone and treats the body;

FIG. 3 is a diagram illustrating elements of an arrayed microwave antenna system according to some embodiments, which is a wearable and treats the head and CNS;

FIG. 4 illustrates elements of an arrayed microwave antenna system according to some embodiments, which is a wearable and treats the body;

FIG. 5 illustrates elements of an arrayed microwave antenna system, which is wearable and treats the head and CNS with an energy dose localized to a lateral position with a focused distribution; and

FIG. 6 illustrates elements of an arrayed microwave antenna system wherein the pattern of their output is effectively directed towards a desired location and energy waves traveling towards undesired locations are quenched.

### DETAILED DESCRIPTION OF THE INVENTION

The construction and usage of embodiments will become readily apparent from consideration of the following specification as illustrated in the accompanying drawings, in which like reference numerals designate like parts, and wherein:

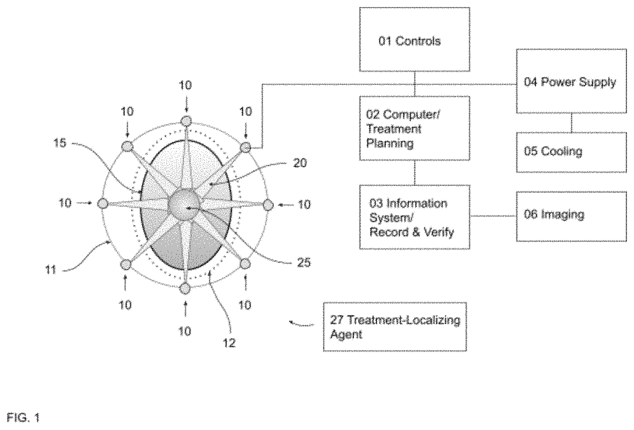

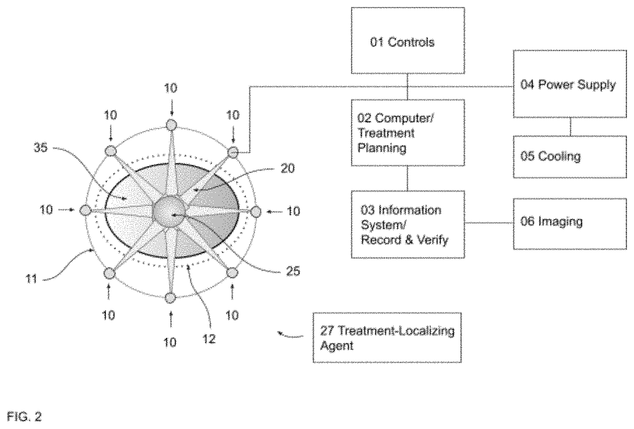

Local and Regional Energy Deposition. In the case of Alzhiemer's disease or other pathologies of the CNS, there are several therapeutic, ameliorating approaches that can be employed around the head 15 as shown in FIG. 1, or body 35 as shown in FIG. 2 with non-ionizing energy 20 of electromagnetic waves in the 300 MHz-300 GHz microwave range, or 300 kHz-300 MHz radio range, or mechanical sound waves in the 0.1 MHz-300 MHz ultrasonic range, including cavitation and boiling histotripsy. In the present invention, non-invasive, i.e., delivered without employing a device or mechanism to cut, puncture or traumatically penetrate the skin, and without an open surgical procedure, external energy 20 can be delivered to a pathological region 25 of the body 35 as shown in FIG. 2, including the head 15 and CNS as shown in FIG. 1, by the spectra and modalities including, but not limited to, the above mentioned heat, pressure and electromagnetic waves and oscillations. With these methods the mechanisms of treating disease can entail, but are not limited to, causing necrosis, induction of apoptosis, induction of heat-shock proteins, folding and unfolding proteins, increased blood, lymphatic or CSF flow, increased permeability of the blood-brain barrier (BBB) to remove toxic by-products and permit entry of beneficial effectors, initiating immune responses (stimulating or suppressing), reducing inflammation and abnormal neural stimulation, and causing euphemistic mechanical, thermal, electromagnetic or chemical interactions. By way of the present invention, non-invasive, external energy sources 10 are aimed to a focus and may employ adjustable phased array electronics to contour different locations, shapes, sizes, frequency and timing, and other treatment related parameters, of the energy output or beams with minimal or no movement of the sources. The energy is thereby concentrated in a targeted volume, enhancing it and made discrete, and therein encompassing of a detrimental lesion. A treatment volume can be maintained or adjusted, spatially and temporally, to cover specific pathology, anatomic and functional structures, or regions of interest. The resulting adjustable radiation field size covers a localized, or regional, internal volume 25 having diameters ranging from nanometers to a plurality of centimeters. A distribution of external energy radiating sources 10 may be in a localized, or circumferential distribution, or a ring 11 or spherical distribution around a patient's head 15, CNS or body 35. The patient's surface is cooled with circulating air or water 12, and may be coated with a conductive gel. The illuminated treatment volume 25 can be maintained or adjusted to cover specific pathology, anatomic and functional structures, or regions of interest. The process and time of treatment delivery may be continuous or continual via a wearable device 19, or fractionated over hours, days or weeks via a stand-alone device 11. Given the variety of energy sources possible with the present invention, they might be employed based on the efficacy of their intensity (controlled by modulating pulse-width) or frequency. The energy sources 10 are connected to a power supply 04 with cooling 05 and utilize computerized controls 01. The power supply 04, can be connected to a microcontroller to provide a means of regulating the sources' intensity and timing. The treatment system includes a computer with treatment planning software (TPS) 02, which can upload images 06 for planning and guidance, and is connected to an information system with record and verify software 03. The TPS consists of a medium storing computer-executable process steps to reconstruct medical images and calculate therapeutic effects of the localized non-ionizing radiation of energy treatment. In some configurations, the system includes a treatment table or chair, capable of movement in three dimensions, and non-invasive or invasive thermometry to measure heat in the tissue volume absorbing the delivered energy 20. In some embodiments, the system of the present invention comprises treatment of diseased tissue 25 with a non-ionizing-radiation, treatment-localizing agent 27, which becomes active in response to received non-ionizing-radiation energy 20. Such non-ionizing-radiation, treatment-localizing agents 27 include, but are not limited to, thermolabile or thermosensitive liposomes, which carry drugs, biologics, immune-modulating compounds, or contrast agents, and microbubbles.

It is an object of this invention to provide a method for depositing energy into a discrete, conforming volume encompassing and customized to a detrimental phase or lesion within a body, and thereby enhancing restoration of control parameters to a normal physiologic range, and thus enabling a healthy transition state. With the present invention, AD might be treated non-invasively, with or without pharmaceuticals, by focusing the output of an annular 11 array of microwave antennas 10, or defocusing the output of an array of HIFU transducers, to raise the temperature of a diseased tissue or volume of the CNS anatomy such as the hippocampus to levels of a fever, e.g., 40° C. or greater for several seconds to 24-hour intervals. In this manner, elevated levels of mis-folded, aggregated or modified proteins, like p-Tau, could be reversed and undone by restoration of lesion or core temperature. The desired effect could be the result of inhibiting pathologic modifications of proteins, e.g., phosphorylation of tau, or supplanting cold inhibition of proteasomes. In addition, local and regional energy deposition or heating to induce hydrodynamic manipulation of intracellular or extracellular fluid is achievable and a preferred form of delivery. For example, a larger fluid volume, greater than a discrete anatomic site such as the CSF, might be targeted to raise its temperature or oscillations and enhance the flow of CSF and the removal of toxic materials and peptide aggregates from the CNS. Heating in this localized or wider instance would enhance protein refolding and enable untangling of protein aggregates, such as Aβ or tau, as well as induce HSPs. The BBB in regions of suspected pathology could also be heated or energized therapeutically to optimize exchange of waste products and toxic metabolites or improve drug passage and delivery. If PD was being treated, the substantia nigra might be selectively heated.

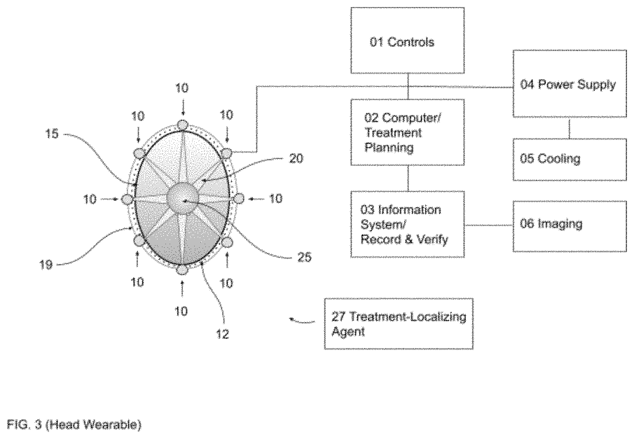

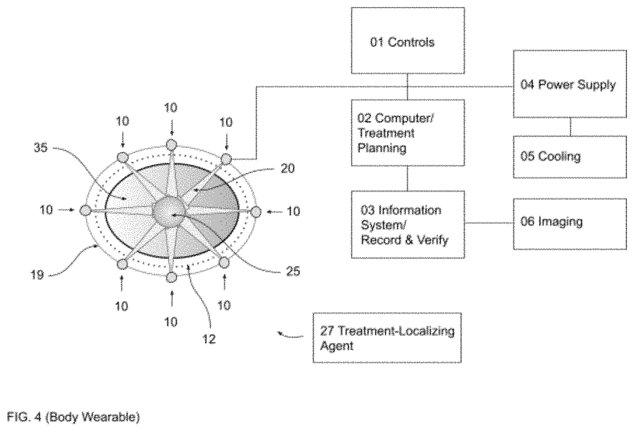

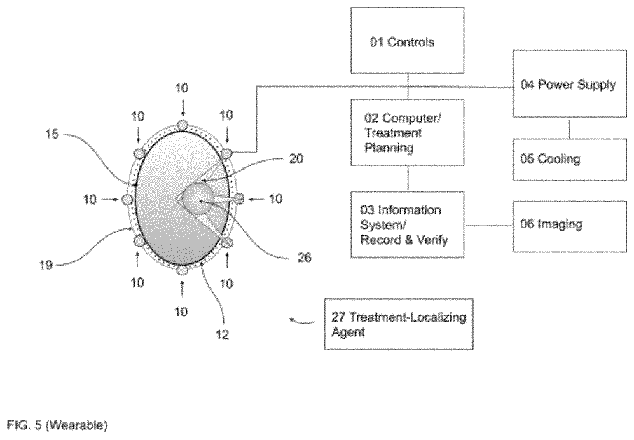

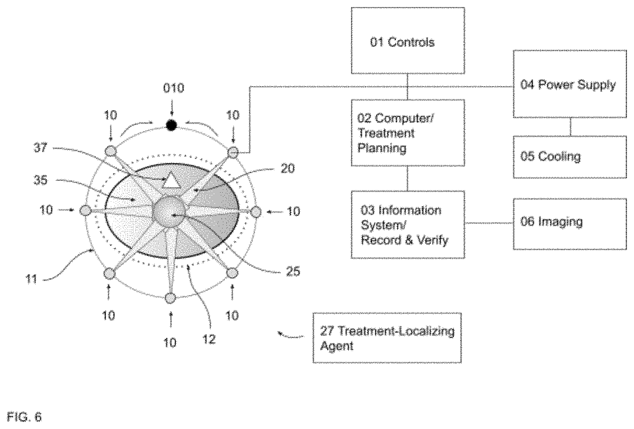

Devices and Wearables. By example, in the present invention, a phased array of energy sources, such as microwave antennas, are aligned in a ring, e.g., stand-alone device 11 around a head 15 as shown in FIG. 1, or body 35 in FIG. 2, or moved by a robot or gantry, or a surface conforming and adhering layout, e.g., wearable device 19 for the head 15 demonstrated in FIG. 3 or body 35 in FIG. 4. The antennas 10, or interfaces for electromagnetic waves propagating in space, and electrons flowing through conductors, are connected to a transmitter. In some examples, the sources or antennas 10 are in a phased array, wherein they do not have to be moved. Rather, to treat pathologic lesions, the non-ionizing energy sources can be electronically or computer controlled to shift and contour their output, e.g., beams, the electromagnetic waves 20 in this instance, in different directions. Thereby, energy dose is delivered discretely and localized to a given depth or lateral position in a body 35 or head 15, illustrated in FIG. 5, with a narrow or focused distribution. The antennas 10 in this invention are distributed in an annular, ringed or spherical arrangement 11 around the body or CNS, such that the pattern of their output 20 or beams 20 is effectively directed towards a desired location, specific or selected volume 26, and energy waves traveling towards undesired locations 37 as shown in FIG. 6, e.g., healthy tissue, are quenched. The overlapping directed waves build up energy deposition in a selected volume 25, which has been delineated beforehand by imaging 06 or functional evaluation, to receive an enhanced dose of the energy or radiation. The effective and deliberate targeting to deliver non-ionizing energy to specific volumes 25 of tissue, locations 26 in FIG. 5, fluid collections, airways or microorganisms inside the body 35 or head 15 is measured and evaluated in real time with imaging, including MRI and MRI thermometry, functional MRI (fMRI) and positron emission tomography (PET), thermometry and other functional metrics. The purpose of the focused and enhanced energy deposition 25 is to reset or disable, including but not limited to: pathologic physiology or structures, nerve pathways, extracellular fluid, misfolded proteins, toxic products or microorganisms, the microbiome, disease-inducing immunity, and abnormal flow of blood, lymph, CSF, metabolites, faulty enzymology along with central and peripheral neural signals. In addition, the delivery of locally augmented energy can be used to enhance: immunity, the presence of chaperone molecules, e.g., heat shock proteins (HSPs, sHSPs), the flow of blood, lymph, CSF, metabolism, metabolites and adaptive enzymology, the delivery of energy-sensitive compounds or pharmaceuticals, carriers, and energy-sensitive vesicles, e.g., thermolabile liposomes or microbubbles.

Delivery Configurations. To deliver effective coverage and dosage with a particular energy 20 such as microwaves to a patient, distribution of emitting antennas 10 around a person facilitates spatial and temporal optimization of the treatment. A stand-alone device with emitting energy sources can move around a patient or have a ringed distribution 11 to achieve this structure. In such a geometry treatments might be given at daily, weekly or monthly intervals. Alternatively, the emitting energy sources can be attached to a patient as a wearable 19 as shown in FIG. 3 and FIG. 4, e.g., cap, bands, clothing, etc., which would permit more frequent pulsing or continual or continuous delivery of effective or initiating energy. The duty cycle with a wearable device 19 could be round-the-clock, scheduled to coincide with circadian rhythms or timed as determined to be most effective by future clinical evaluation. The duration of the heating can be seconds when high flux is employed to months when lower flux is delivered. The longer delivery times can be achieved by the device configuration that is worn by the patient, i.e., wearables 19, which can continually or continuously apply energy 20 to the zone of interest or abnormality 26.

Protein Aggregation. Misfolded proteins can aggregate and are implicated in a range of pathology including AD, PD, prion disease and cancer. The native state of the protein structure is maintained by non-covalent and disulfide bonds. These include ionic and van der waals forces. Genetic mutations, aberrant synthesis and environmental factors, e.g., abnormal temperature, pH, oxidation or aging, can destabilize bonding and native conformation resulting in protein unfolding. Unfolded or misfolded proteins, if not refolded or degraded, are susceptible to aggregation, wherein exposed hydrophobic sections may interact between proteins. These aggregates may take different forms including protein aggregates, amyloid fibrils, tangles, oligomers and degraded peptides. Refolding can take place in the presence of molecular chaperones, such as HSPs, while degradation entails elimination via the ubiquitin-proteasome system, or autophagy in lysosomes. Prions are resistant to ionizing radiation, and they do not stimulate humoral immunity, consistent with their being immunologically inert, self-encoded proteins.

HSPs, Chaperone Induction via Directed Heating. The forces by environmental stresses, e.g., increased temperature (38°-43° C. or greater), and reduced pH (<7.4), acting to alter protein conformation are counteracted in many biological systems, including mammals, by chaperone molecules. For example, HSPs engage as chaperones of unfolding proteins, and are important for refolding denatured proteins, as well as participating in antigen presentation to dendritic cells (antigen presenting cells, APCs), and eventually lymphocytes. In addition, HSPs have a role in reversing aggregation and promoting fragmentation of large polymers formed after coalescence of misfolded proteins. Therefore, when applied with therapeutic intent by way of the present invention, focused, enhanced and localized heating or other energy deposition in diseased tissue, can be employed to induce refolding itself or induce chaperone molecules, e.g., HSPs, for refolding, antigen presentation, or reversing aggregation and promoting fragmentation of large polymers formed after coalescence of misfolded proteins. The effects on detrimental peptide elements include but are not limited to: i) refolding misfolded proteins, fibrils, tangles and protein aggregates, ii) enhancing degradation and elimination of misfolded proteins, fibrils, tangles and protein aggregates, and iii) enhancing immune processing via antigen presentation of misfolded proteins, fibrils, tangles and protein aggregates, as well as, enhancing fluid circulation (see below), to repair and remove toxic metabolites in a body, including the CNS, the peripheral nervous system, the musculoskeletal system, the genitourinary system, the gastrointestinal system, the integument and other anatomy.

Non-Ionizing-Radiation, Treatment-Localizing and Enhancing Agents. Thermolabile or thermosensitive liposomes (TSLs) can enhance and localize delivery of compounds, drugs and/or imaging agents via heat-induced release at a target tissue. After intravascular infusion, the liposomes are selectively ruptured at a desired location by warming the site 25 to temperatures in the upper range of tolerated fevers, e.g., 40°-43° C. Applications include but are not limited to: imaging and therapy of cancer, localized infection, inflammatory pathologies and damaged anatomy. The chemical composition and function of TSLs have evolved (along with the heating modalities and devices employed). For example, early thermolabile liposomes were unstable because they used a single-chain lipid, but allowed quick drug release in response to a heat trigger. Better performance has been achieved with a two-chain lipid, which uses a novel phospholipid, DPPG2 (see U.S. Pat. No. 9,980,907). This TSL achieves both quick drug delivery under heat trigger and prolonged circulation absent applied heat. Its contents comprise doxorubicin, gemcitabine or a gadolinium-based contrast agent (Thermosome GmbH, Planegg, Germany). In addition to conventional chemotherapy drugs or radiographic imaging agents comprising the contents of a TSL, the present invention can use TSLs carrying immunotherapy or immune-modulating agents, including but not limited to cytokines such as, human granulocyte-macrophage colony-stimulating factor (hGM-CSF), e.g., sargramostim, Leukine (Partner Therapeutics, Inc., Lexington, Mass.). There are no presently marketed TSLs capable of carrying biologic agents or cytokines, such as GM-CSF, or recombinant proteins. When an immunotherapeutic agent is delivered with TSLs in the present invention, it is locally released by concentrating a preponderance of TSLs within a discrete targeted volume, preferably pre-treated with a modality to induce necrosis, and accessible to blood flow or cerebrospinal fluid distribution, that is heated by the present invention with concentric, converging electromagnetic energy beams in the microwave range, 300 megahertz (MHz) to 300 gigahertz (GHz). Thus, this enables systemic infusion of an immunomodulator, or other compound, but limits its interactions to a localized volume of interest. Targeting a lesion within a body by employing volumetrically discrete energy dose deposition to beneficially manipulate the lesion with a therapeutic compound therein, as recited in the present invention, compromises a treatment-localizing and enhancing agent 27. This is important in the case of inducing beneficial therapeutic effects, e.g., anti-tumor immunity with GM-CSF, where localized cytokine delivery to necrotic debris is thought to be efficacious and has been described (U.S. Pat. No. 7,481,758), while systemic distribution is not desirable since it can induce immune suppression and other side effects (Weil and Morris, unpublished). In addition, repeating mechanical waves and beams generated by ultrasound or highly-focused ultrasound (HIFU) with frequencies ranging from 0.5 mHz to 30 GHz can be brought to a focus to heat tissue and generate a heat zone to trigger the release of agents carried by TSLs, in addition to being effective modality for inducing necrosis.

Hydrodynamic Manipulation. The non-invasive delivery of localized heat to a volume of fluid, tissue or an organ involved in pathology secondary to protein aggregates offers the opportunity to invoke several reparative pathways. Heating the CSF results in the enhancement of its movement through the cerebrospinal space, which includes the subarachnoid space (between the arachnoid mater and pia mater) and the ventricular system around and inside the spinal cord and brain, including the choroid plexus, arachnoid villi and granulations, 3rd and 4th ventricles of the brain, cisterns, sulci, cauda equina and central spinal canal. Thereby it will make possible optimization of CSF flows, which are coupled to blood-flow and blood-oxygenation changes during slow-wave (non-REM) sleep, and thereby enhance removal of pathologic protein aggregates and other toxic metabolites.

Non-Healing Fractures and Wounds, Lung, Ophthalmology and Dentistry. Applied electrical stimulation is established clinically for jumpstarting repair of non-healing bone fractures and to induce wound healing following surgery or in the setting of impaired wound healing as seen in diabetic patients. The present invention of arraying microwave antennas around a pathologic site to focus and enhance microwave range energy can be employed in place of the conventional electric field stimulation in these settings. A non-healing fracture, or non-healing wound could be energized in this fashion to enhance recovery. Infection with microorganisms, including but not limited to, bacteria and associated biofilms, mycoplasma, viruses, fungi, archaea, amoebae and prions, and protein misfolding or unfolding abnormalities in the proteostasis network (PN), can similarly call up responses like a wound, which require, i) an enhanced immune response, ii) blunting of bacterial signaling, e.g., bacteria employ “quorum sensing” (by way of molecular messengers to coordinate growth and optimize uptake of nutrients and protective factors), and iii) clearance of cellular debris and biofilms. The implementation of the present invention and resulting enhancement of immunity, vascular and lymphatic flow can expedite the recovery from such infections and contagion throughout the body, including but not limited to the lungs, liver, gut, musculoskeletal system, eyes and CNS. Moreover, homeostasis of the microorganisms and integrity of the nose, sinuses, mouth, gums and teeth is maintained via pathways that can be rectified with the present invention. Thus, a degrading impact of periodontal disease on the CNS, e.g., AD or PD or neuroinflammation and prion-like mechanisms, might be corrected, and both oral and mental function restored.

Viruses. High temperature induced by transferring energy from outside a virally or microbe infected cell, tissue, organ or body, into a volume, partly or entirely encompassing and localizing to the affected structure, reduces viral replication and function, such as in the case of heating influenza virus in a range of 39°-43° C. Heating subcellular organelles, cells, tissue and anatomic sites renders them more resistant to viral penetration and escape, for example, by raising endosomal pH and inhibiting IL-6, a mediator of viral reproduction. Moreover, heat shock protein promoters, e.g., those of Hsp70, when heated to 39°-43° C., or greater, initiate global gene expression of antiviral proteins to inactivate or counteract virality.

What is claimed is:

1. A system comprising:
a. a distribution of energy radiating sources, which are external and around a body, or head and central nervous system, to treat and image pathologic lesions;
b. wherein the radiated energy is non-ionizing, non-ablating electromagnetic or mechanical;
c. wherein the radiated energy comprises a combination of non-ionizing output or beams selected from the group consisting of microwave radiation, radiofrequency radiation, long radio waves, laser emissions, visible, ultraviolet and solar light, infrared and far infrared light, thermal chambers, or incubators, diathermy or high-frequency electric currents, pulsed, mechanical or acoustic waves, ultrasound and high-intensity focused ultrasound (HIFU), heat from nano-devices and warm water;
d. wherein the energy is delivered non-invasively without employing a device or mechanism to cut, puncture or traumatically penetrate skin or other surface, and without an open surgical procedure;
e. wherein the energy-emitting sources are aligned in a localized, or circumferential, or ring or spherical distribution around a patient, as a stand-alone arrangement, or moved by a robot or gantry, or in a surface conforming and adhering layout, as a wearable;
f. a phased array of the non-ionizing energy sources, electronically or computer controlled, to shift and contour their output or beams in different directions without moving the sources, for discrete and localized delivery of energy dose to a given depth or lateral position in the body with a narrow or focused distribution;
g. wherein the resulting contoured energy output or beams are concentric, converging electromagnetic energy beams and deposit energy dose in a localized volume of interest to treat pathologic tissue;
h. wherein the conforming volume is discrete, encompassing and customized to a detrimental phase or lesion within the body, thereby enhancing restoration of control parameters to a normal physiologic range and thus enabling a healthy transition state;
i. electronic or computerized controls for source and system operation with a connected power supply with cooling;
j. imaging for planning and guidance, a computer with treatment planning software, which can upload images for planning and guidance, and is connected to an information system with record and verify software;
k. a medium storing computer-executable process steps to reconstruct medical images and calculate therapeutic effects of the localized non-ionizing radiation of energy treatment;
l. a treatment table or chair capable of movement in three dimensions;
m. a non-ionizing-radiation, treatment-localizing agent to treat diseased tissue in response to received radiation of energy; and
n. non-invasive or invasive thermometry.

2. A device comprising:
a. a distribution of energy radiating sources, which are external and around a body, or head and central nervous system, to treat and image pathologic lesions;
b. wherein the radiated energy is non-ionizing, non-ablating electromagnetic or mechanical;
c. wherein the radiated energy comprises a combination of non-ionizing output or beams selected from the group consisting of microwave radiation, radiofrequency radiation, long radio waves, laser emissions, visible, ultraviolet and solar light, infrared and far infrared light, thermal chambers, or incubators, diathermy or high-frequency electric currents, pulsed, mechanical or acoustic waves, ultrasound and high-intensity focused ultrasound (HIFU), heat from nano-devices and warm water;
d. wherein the energy is delivered non-invasively without employing a device or mechanism to cut, puncture or traumatically penetrate skin or other surface, and without an open surgical procedure;
e. wherein the energy-emitting sources are aligned in a localized, or circumferential, or ring or spherical distribution around a patient, as a stand-alone arrangement, or moved by a robot or gantry, or in a surface conforming and adhering layout, as a wearable;
f. a phased array of the non-ionizing energy sources, electronically or computer controlled, to shift and contour their output or beams in different directions without moving the sources, for discrete and localized delivery of energy dose to a given depth or lateral position in the body with a narrow or focused distribution;
g. wherein the resulting contoured energy output or beams are concentric, converging electromagnetic energy beams and deposit energy dose in a localized volume of interest to treat pathologic tissue;
h. wherein the conforming volume is discrete, encompassing and customized to a detrimental phase or lesion within the body, thereby enhancing restoration of control parameters to a normal physiologic range and thus enabling a healthy transition state;
i. electronic or computerized controls for source and system operation with a connected power supply with cooling; and
j. a medium storing computer-executable process steps to reconstruct medical images and calculate therapeutic effects of the localized non-ionizing radiation of energy treatment.

3. The device according to claim 2, further comprising:
a. adjustable phased array electronics to contour different locations, shapes, sizes, frequency and timing, and other treatment related parameters of the energy output or beams;
b. wherein a treatment volume can be maintained or adjusted to cover specific pathology, anatomic and functional structures or regions of interest;
c. wherein a resulting adjustable radiation field size covers an internal treatment volume having diameters ranging from nanometers to a plurality of centimeters; and
d. wherein a medium storing computer-executable process steps adjusts a mechanism's radiation emission for treatment and imaging.

4. The device according to claim 2, further comprising:
a. antennas or interfaces for electromagnetic waves propagating in space, and electrons flowing through conductors, are connected to a transmitter;
b. wherein the antennas may or may not be in a phased array; and
c. overlapping directed waves build up energy deposition in a selected volume, which has been delineated beforehand by imaging or functional evaluation, to receive an enhanced dose of energy or radiation.

5. A method comprising:
a. effective and deliberate targeting to non-invasively deliver non-ionizing, non-ablating electromagnetic or mechanical energy to specific volumes of tissue, locations, fluid collections, airways or microorganisms inside a body or head, wherein the energy targeting is measured and evaluated in real time with imaging, thermometry or other functional metrics;
b. wherein the non-ionizing energy output is effectively directed towards a desired location, specific or selected volume, and energy waves traveling towards undesired locations, such as healthy tissue, are quenched;
c. obtaining an original condition, state, conformation, functionality or phase in the body by introducing adequate localized energy to an altered process, network or system;
d. wherein the energy is focused, enhanced and localized energy, and reverses prion-like mechanisms, wherein misfolded, or unfolded proteins, which may be unaggregated or aggregated, are subjected to a permissible temperature or energy dose, thereby enabling restoration to their native, normal conformation;
e. counteracting environmental stresses and forces, which alter protein conformation, consisting of but not limited to, increased temperature, 38°-43° C. or greater, and reduced pH, <7.4, by inducing chaperone molecules, such as HSPs, for refolding denatured proteins and antigen presentation to dendritic cells and reversing aggregation;
f. employing focused, enhanced and localized heating or other energy deposition to induce refolding itself or chaperone molecules for refolding or antigen presentation and reversing aggregation;
g. inducing an effect in a diseased tissue wherein the effects comprise a combination of refolding, reversing aggregation, promoting fragmentation, enhancing degradation, elimination, enhancing immune processing and antigen presentation of peptide elements, each having a detrimental impact selected from the group consisting of misfolded proteins, their large polymers, fibrils, tangles and protein aggregates; and
h. wherein the induced effects of focused, enhanced and localized energy deposition further compromise a combination of enhancing fluid circulation to repair and remove toxic metabolites in bodies, each having a diseased tissue selected from the group consisting of the CNS, the peripheral nervous system, the musculoskeletal system, the genitourinary system, the gastrointestinal system, the integument and other anatomy.

6. The method according to claim 5 wherein:
a. distributing energy radiating sources, externally and around a body, including head and central nervous system, to treat and image pathologic lesions;
b. radiating an energy that is non-ionizing, non-ablating electromagnetic or mechanical;
c. wherein the radiated energy comprises a combination of non-ionizing output or beams selected from the group consisting of microwave radiation, radiofrequency radiation, long radio waves, laser emissions, visible, ultraviolet and solar light, infrared and far infrared light, thermal chambers, or incubators, diathermy or high-frequency electric currents, pulsed, mechanical or acoustic waves, ultrasound and high-intensity focused ultrasound (HIFU), heat from nano-devices and warm water;
d. delivering the energy non-invasively without employing a device or mechanism to cut, puncture or traumatically penetrate skin or other surface, and without an open surgical procedure;
e. aligning the energy-emitting sources in a localized, or circumferential, or ring or spherical distribution around a patient, as a stand-alone arrangement, or moved by a robot or a gantry, or in a surface conforming and adhering layout, as a wearable;
f. employing a phased array of the non-ionizing energy sources, electronically or computer controlled, to shift and contour their output in different directions without moving the sources, for discrete and localized delivery of energy dose to a given depth or lateral position in the body with a narrow or focused distribution;
g. contouring energy output or beams to concentric, converging electromagnetic energy beams and depositing energy dose in a localized volume of interest to treat pathologic tissue;
h. controlling the sources electronically or with computers, and operating with a connected power supply with cooling; and
i. depositing energy dose locally by shifting and contouring energy output, or beams, for treatment comprising a combination of pathologic lesions, each having a detrimental impact selected from the group consisting of infectious agents, protein misfolding, temperature and pH imbalances; cancer, cancerous and benign tumors and mass effects, demyelination, autoimmunity, loss of immunity, loss of normal mitochondrial and other organelle function; loss of neurotransmitters and neurotransmitter-degrading enzymes, vascular abnormalities and abnormal cerebral blood flow, aneurysms, atherosclerosis, arteriosclerosis, hemorrhage, blood clots, microvascular angiopathy, retinopathy, excess hemoglobin A1c, diabetic angiopathy; abnormalities of the cerebrospinal fluid (CSF) and its production and movement, hormonal imbalances, trauma and traumatic encephalopathy, mental illnesses, neuroses, addiction, depression, mania, schizophrenia and psychoses.

7. The method according to claim 6 further comprising:
a. focusing the output of an array of microwave antennas to raise the temperature of a diseased volume of anatomy to levels in a range of 38°-43° C., or of a fever such as 40° C. or greater, for several seconds to 24-hour intervals;
b. wherein the array is annular in a stand-alone or wearable layout;
c. wherein the anatomy comprises a combination of sites selected from the group consisting of CNS, hippocampus, substantia nigra, blood-brain barrier (BBB), CSF, cranial nerves and extracranial vagus nerve fibers;
d. targeting a lesion within a body by employing volumetrically discrete energy dose deposition to beneficially manipulate lesions;
e. wherein the conforming, discrete volume is encompassing and customized to a detrimental phase or lesion within the body, thereby enhancing restoration of control parameters to a normal physiologic range and thus enabling a healthy transition state;
f. wherein a resulting adjustable radiation field size covers an internal treatment volume having diameters ranging from nanometers to a plurality of centimeters;
g. inducing hydrodynamic manipulation of intracellular or extracellular fluid with local and regional energy deposition or heating; and
h. repairing, reversing or removing toxic materials and peptide aggregates from the CNS.

8. The method according to claim 7 further comprising:
a. arraying microwave antennas around a pathologic site to focus and enhance microwave range energy;
b. treating by applying energy to pathology selected from the group consisting of non-healing wounds, infections with microorganisms, bacteria, mycoplasma, viruses, fungi, archaea, amoebae, prions, protein misfolding, unfolding abnormalities in the proteostasis network, abnormalities in the microbiome, microorganism or bacterial signaling and quorum sensing, and biofilms;
c. enhancing immunity, vascular and lymphatic flow to expedite recovery from infections and contagion throughout portions of a body selected from a group comprising the lungs, liver, gut, musculoskeletal system and CNS, the vagus, olfactory, and ophthalmic nerves and eyes and other cranial nerves, the nose, sinuses, mouth, gums and teeth; and
d. modifying inflammatory reflexes linking metabolism to immune function.

9. The method according to claim 6 wherein:
a. treating diseased tissue with a non-ionizing-radiation, treatment-localizing agent, wherein the agent becomes active in response to received non-ionizing-radiation energy;
b. wherein the non-ionizing-radiation, treatment-localizing agents are selected from the group consisting of thermolabile or thermosensitive liposomes, which carry drugs, biologics, immune-modulating compounds, contrast agents; and microbubbles; and
c. targeting a lesion within the body by employing volumetrically discrete energy dose deposition to beneficially manipulate the lesion with a therapeutic compound therein.

10. The method according to claim 5 wherein:
a. defocusing the output of an array of HIFU transducers to raise the temperature of a diseased volume of anatomy to levels in a range of 38°-43° C., or of a fever such as 40° C. or greater, for several seconds to 24-hour intervals;
b. wherein the array is annular in a stand-alone or wearable layout;
c. wherein the anatomy comprises a combination of sites selected from the group consisting of CNS, hippocampus, substantia nigra, blood-brain barrier (BBB), CSF, cranial nerves and extracranial vagus nerve fibers;
d. targeting a lesion within a body by employing volumetrically discrete energy dose deposition to beneficially manipulate lesions;
e. wherein a resulting adjustable radiation field size covers an internal treatment volume having diameters ranging from nanometers to a plurality of centimeters;
f. inducing hydrodynamic manipulation of intracellular or extracellular fluid with local and regional energy deposition or heating; and
g. repairing, reversing or removing toxic materials and peptide aggregates from the CNS.

11. The method according to claim 5 further comprising:
a. transferring energy from outside a virally or microbe infected cell, tissue, organ or body, into a volume, partly or entirely encompassing and localizing to an affected structure thereof;
b. wherein a radiated energy comprises a combination of non-ionizing output or beams selected from the group consisting of microwave radiation, radiofrequency radiation, long radio waves, laser emissions, visible, ultraviolet and solar light, infrared and far infrared light, thermal chambers, or incubators, diathermy or high-frequency electric currents, pulsed, mechanical or acoustic waves, ultrasound and high-intensity focused ultrasound (HIFU), heat from nano-devices and warm water;
c. delivering the energy non-invasively without employing a device or mechanism to cut, puncture or traumatically penetrate skin or other surface;
d. inducing high local temperature in the encompassing volume and affected structure, in a range of 38°-43° C. or greater, therein heating subcellular organelles, cells, tissue and anatomic sites and rendering them more resistant to viral penetration and escape;
e. wherein the high local temperature raises endosomal pH and inhibiting IL-6, a mediator of viral reproduction;
f. wherein the high local temperature induces heat shock protein promoters, such as those of Hsp70, initiating global gene expression of antiviral proteins, thereby inactivating or counteracting virility; and
g. wherein the transferred energy reduces viral replication and function.

12. The method according to claim 5 wherein:
a. focusing and enhancing non-ionizing energy deposition in a body, wherein energy output is effectively directed towards a desired location, specific or selected volume;
b. reversing or rectifying prion-like mechanisms in the volume, wherein misfolded, or unfolded proteins, which may be unaggregated or aggregated, are subjected to a permissible temperature or energy dose, thereby enabling restoration to their native, normal conformation;
c. obtaining an original condition, state, conformation, functionality or phase in the body by introducing adequate localized energy to a deleterious altered process, network or system and thereby overcoming an undesired transition;
d. wherein depositing energy into the discrete, conforming volume encompassing and customized to a detrimental phase or lesion within the body, thereby enhances restoration of control parameters to a normal physiologic range and enabling a healthy transition state;
e. wherein elevated levels of mis-folded, aggregated or modified proteins, including p-Tau, are reversed and undone by restoration of lesion or core temperature; and
f. wherein inhibiting pathologic modifications of proteins, including phosphorylation of tau or supplanting cold inhibition of proteasomes, yields a desired effect.
